(19) United States
(12) Patent Application Publication
(10) Pub. No.: US 2004/0024392 A1
(43) Pub. Date: Feb. 5, 2004

(54) Apparatus and method for cryosurgery

(76) Inventors: James D. Lewis, Flagstaff, AZ (US); David J. Myers, Camp Verde, AZ (US)

Correspondence Address: David J. Johns, W. L. Gore & Associates, Inc., 551 paper Mill Road, P.O. Box 9206, Newark, DE, 19714-9206, US

(21) Appl. No.: 10/213,114

(22) Filed: Aug. 5, 2002

(51) Int. Cl. A61B 18/18

(52) U.S. Cl. 606/22; 604/523

(57) ABSTRACT

An improved apparatus for delivery of cryosurgery fluid in a surgical or other medical environment is disclosed. The preferred apparatus comprises a multiple-layered expanded polytetrafluoroethylene conduit that has a low profile, has low thermal conductivity, and provides exceptional flexibility. A wide variety of treatment instrumentalities may be employed on the end of the conduit to provide medical treatments ranging from direct topical application of cryosurgery fluid to open or closed-system surgical or endosurgical uses.

### BACKGROUND OF THE INVENTION

1. Field of the Invention

The present invention relates to apparatus and method for use in handling and controlled delivery of cold fluids. In particular, the present invention relates to apparatus and methods for using cold fluids in a medical environment.

2. Description of Related Art

Cold to extremely cold fluids, both as gases and liquids, are used in various surgical procedures today. Typically, such fluids, such as liquid nitrogen or liquid air, are applied in a focused manner to a patient's tissue to kill unwanted cells, such as cancerous tissue, or to freeze tissue or an entire organ for later use. Such uses include procedures referred to as “cryoablation” and “cryotherapy.” Applications for these materials include topical use to treat skin defects, ablation of cancer or other malfunctioning cells, cardiac ablation, prostate and inter-uterine treatments, and intravascular treatments, such as for prevention of stenosis.

In cryosurgery in its simplest form, such as for topical applications, physicians generally use a metal nozzle directly attached to a canister of cryogenic fluid. The nozzle sprays cryogenic fluid onto the patient to freeze the target site.

Other cryosurgical procedures are performed with often complicated cryosurgery apparatus involving very high-pressure fluids and or refrigerants that are conveyed to a delivery instrumentality, often remote from the fluid source and sometimes deep within a patient's body. These modalities can pose significant risk to the patient and/or the medical staff in the event of a failure. The devices operate by directly delivering fluids through a tube or catheter to chill a probe or other instrumentality at the distal end. Devices of this type are sometimes known as “cryostats” or “cryocoolers” and use a Joule-Thompson cooling mechanism that takes advantage of the fact that many gases when rapidly expanded become extremely cold. In devices of this type very high-pressure gases such as argon or nitrogen are piped at or near ambient temperature down a delivery catheter to a surgical probe or instrumentality. The gas is then expanded through a nozzle to creat a very cold condition that is typically below −120° C.

Another type of cold-surgery device utilizes liquid refrigerants that are piped down the catheter at or near ambient temperatures. Once the refrigerant reaches the distal instrumentality, a phase change of the refrigerant occurs which produces a very cold localized atmosphere or instrumentality.

None of these current procedures is entirely satisfactory due to the complexity and inherent danger of high-pressure apparatus. It is believed that a simple low-pressure system that pipes cryogenic fluid through a catheter directly to the distal instrumentality would be an advantage. There are numerous medical procedures that might benefit from use of cryogenic fluid, but cannot employ such fluids because current delivery apparatus will not accommodate the physical constraints of the procedures. For example, it is believed that numerous endoscopic or endoluminal procedures could benefit from the use of very cold fluids, but currently available thick, short, and inflexible conduits cannot provide suitable means for cryogenic fluid delivery. Additionally, the thermal inefficiency of some of the existing conduits make them either too cold to handle with bare hands or to apply against unprotected tissue, and/or incapable of maintaining a cryogenic fluid in a liquid state from a storage container to a distant delivery site.

Accordingly, it is a primary purpose of the present invention to provide a conduit to deliver fluids in cryosurgery procedures that addresses the shortcomings of current cryosurgery apparatus and allows for a simple and more efficient delivery of cold fluids to a wider variety of treatment sites.

It is a further purpose of the present invention to provide cryosurgery fluid delivery apparatus that has a low profile, is thermally efficient, and provides flexibility.

These and other purposes of the present invention will be better appreciated through review of the following specification.

### SUMMARY OF THE INVENTION

The present invention is improved apparatus and method for delivery of cold fluids during cryosurgery to a medical treatment site. The present invention employs a very low profile expanded polytetrafluoroethylene (PTFE) tube to deliver cryosurgery fluid, either a gas or a liquid or both, from a cryosurgery fluid source to a treatment site. Due to the unique properties of the conduit employed with the present invention, the tube has excellent thermal properties, allowing the conduit to transport extremely cold fluids, and especially cryogenic liquids, over an extended length with minimal profile. This can be accomplished without undue loss of cryosurgery fluid and without the conduit becoming too cold to handle. The conduit of the present invention is flexible even at extreme cold temperatures, allowing the conduit to be bent 90 degrees or more, with a small (e.g., less than about 25 mm) radius of curvature, while carrying cryosurgery fluids without compromising the fluid retention properties of the conduit. Even more notable, the conduit is flexible enough that it can be thoroughly kinked so as to cease cryosurgery fluid flow therethrough and then released to restore normal fluid flow with no structural failure of the tube.

In one embodiment of the present invention, it comprises a medical apparatus having: a flexible conduit of expanded polytetrafluoroethylene; a fitting on the conduit adapted to attach the conduit to a source of liquid cryosurgery fluid; and an instrumentality on the conduit adapted to employ the cryosurgery fluid at a treatment site. The conduit is adapted to convey cryosurgery fluid from the source of liquid cryosurgery fluid to the instrumentality. Preferably, the conduit includes at least two lumens therethrough, a first lumen for delivery of cryosurgery fluid to the treatment instrumentality (e.g., a sealed metal or polymer probe adapted to be cooled to cryosurgery temperatures) and a second lumen to remove cryosurgery fluid from the treatment site for discharge away from the patient.

One of the benefits of the present invention is that it can successfully deliver cryosurgery fluids, and especially cryosurgery liquids, at relatively low delivery pressures. In this way, cryosurgery fluids can be delivered with the present invention in a manner that is both safer and more efficient than previous cryosurgery fluid delivery apparatus.

These and other benefits of the present invention will be appreciated from review of the following description.

### DESCRIPTION OF THE DRAWINGS

The operation of the present invention should become apparent from the following description when considered in conjunction with the accompanying drawings, in which:

FIG. 1 is a top elevation view of one embodiment of delivery apparatus of the present invention, including a fitting on a first end for attachment to a cryosurgery fluid source and an opening on a second end for delivery of a cryosurgery fluid;

FIG. 2 is an enlarged isometric view of one end of one embodiment of a conduit of the present invention;

FIG. 3 is a cross section view of a segment of the conduit shown in FIG. 2;

FIG. 4 is a top elevation view of another embodiment of delivery apparatus of the present invention, the apparatus comprising a dual lumen conduit, a fluid source fitting and an exhaust port on a first end of the conduit, and treatment instrumentality on a second end of the conduit, wherein the second end of the dual lumen conduit is illustrated through a cut-away in the illustration of the instrumentality;

FIG. 5 is a three-quarter isometric view of another embodiment of a coaxial lumen conduit of the present invention attached to another embodiment of a treatment instrumentality;

FIG. 6 is a three-quarter isometric view of a treatment instrumentality of the present invention comprising a probe being attached to a first conduit for delivery of a cryosurgery fluid and a second conduit for removal of cryosurgery fluid;

FIG. 7 is a three-quarter isometric view of another embodiment of a second end of a conduit of the present invention, wherein the conduit is provided with a closed end and the conduit is adapted to provide controlled weep of cryosurgery fluid at its second end;

FIG. 8 is a top elevation view of still another embodiment of the apparatus of the present invention, comprising a conduit with a cryosurgery fluid source fitting on its first end and a cryosurgery fluid delivery needle on its second end;

FIG. 9 is a schematic representation of delivery apparatus of the present invention illustrating a conduit of the present invention attached to a dewar containing cryosurgery fluid, and pressure source attached to the dewar to facilitate delivery of fluid from the dewar through the conduit;

FIG. 10 is a three-quarter isometric view of a further embodiment of a conduit of the present invention including a braided cover applied thereto;

FIG. 11 is a three-quarter isometric view of a still further embodiment of a conduit of the present invention including an electrical conductor embedded therein;

FIG. 12 is a front elevation view of a tube undergoing a 180° bending with a relatively wide radius of curvature in an initiation of a flow-stopping kink test described herein;

FIG. 13 is a front elevation view of a tube undergoing a 180° bending with a flow-stopping kink achieved in a flow-stopping kink test described herein; and

FIG. 14 is a schematic representation of test apparatus for determining relative thermal efficiencies of cryosurgery tubes in accordance with Example 11.

### DETAILED DESCRIPTION OF THE INVENTION

The present invention comprises apparatus that provides improved delivery and use of cryosurgery fluids, both liquids and gases, for use in a variety of surgical applications.

For the purpose of the present application, all medical uses whereby cold (below about 0° C.) to very cold (below about −40° C.) to extremely cold (below about −80° C.) temperatures are applied to tissue to destroy or treat cells at temperatures well below freezing are referred to as “cryosurgery.” The term “cryosurgery fluids” as used herein refers to any gas or liquid that can be used in a cryosurgery procedure to establish appropriately cold temperatures at a treatment site. This includes treatments with a variety of “cryogenic” fluids, such as compressed gaseous nitrogen, which is commonly applied at temperatures of −50° C. or less, and liquid nitrogen, which is commonly applied at temperatures of −100 to −150° C. or less.

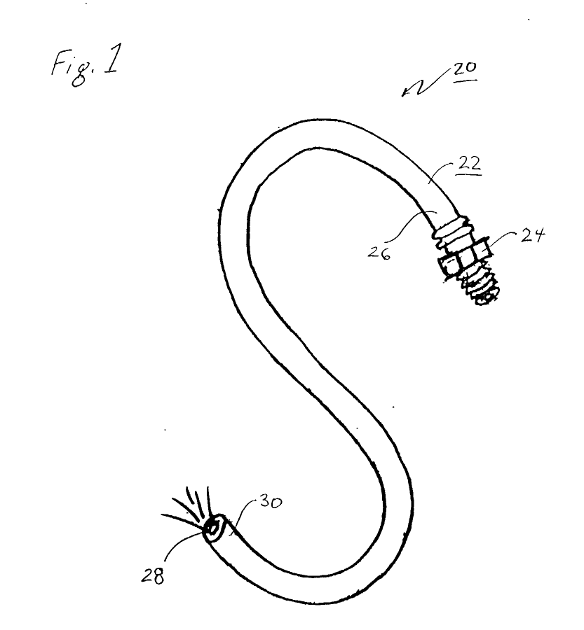

FIG. 1 illustrates one embodiment of delivery apparatus 20 of the present invention. The delivery apparatus 20 comprises a conduit 22 that has a fitting 24 attached to its first end 26 and a delivery opening 28 on its second end 30. The fitting 24 is adapted to be attached to a cryosurgery fluid source, such as a pressured dewar described below. The delivery opening 28 may be used alone as an instrumentality to deliver cryosurgery fluid directly to a treatment site. Alternatively, the delivery opening 28 may be proportioned to attach to an additional instrumentality (such as a probe, nozzle, needle, or balloon) for delivering cryosurgery fluid for use at the treatment site (i.e., either by direct application of the cryosurgery fluid to the tissue to be treated (such as by a nozzle or open-end needle) or by cooling of the instrumentality (such as a probe, balloon, or close-end needle) with cryosurgery fluid that is then removed from the treatment site through a return fluid line.

The conduit 22 is preferably formed from a polymer material that has a number of desired properties, including: excellent thermal properties, allowing the conduit to transport cryosurgery fluid, and especially cryosurgery liquids, over an extended length with minimal overall profile and with minimal heat capacity; and good flexibility, allowing the conduit to be easily bent 90 degrees or more, with a small (e.g., less than 25 mm) radius of curvature, while carrying cryosurgery liquids without compromising the fluid retention properties of the conduit. As can be seen in FIG. 1, the conduit 22 is highly flexible, allowing it to be readily bent into a variety of shapes, such as the serpentine shape shown. This flexibility of the conduit enables it to be readily inserted into the body as well as easily manipulated by the medical staff before, during, and after a procedure.

The preferred polymer material for the conduit comprises an expanded polytetrafluoroethylene (PTFE) characterized by a microscopic structure of polymeric nodes and fibrils. Expanded PTFE, which can be made to be either permeable or impermeable through its wall surface, has excellent stability at cryogenic temperatures (typically as low as −150 to −196° C. or colder) without significant loss of flexibility, low heat capacity, and the ability to withstand extreme bending and kinking without cracking or leaking. Additionally, expanded PTFE has demonstrated excellent temperature insulative properties, allowing even very thin tubes (e.g., with a wall thickness of 0.5 to 1 mm or less) to transport liquid cryogenic fluids while exhibiting an outside temperature that can be handled for brief periods of time without insulative gloves and that will not damage tissue or body fluids coming in contact with the conduit during the time required for a brief surgical procedure. Expanded PTFE also lends itself to ready inclusion of additional insulative layers around the conduit as desired. Finally, millions of successful long-term implants have demonstrated that expanded PTFE has excellent bio-compatibility, making it suitable for use with numerous medical procedures.

For use as a conduit 22 of the present invention, a tube may be constructed with expanded PTFE films and tubes and polymer films such as fluorinated ethylene propylene (FEP), perfluoroalkoxy polymer (PFA), etc. The porous expanded PTFE components have a multiplicity of nodes and fibrils and may be made as taught by U.S. Pat. Nos. 3,953,566, 4,187,390, 5,814,405, or 5,476,589.

To construct a tube of the present invention, a mandrel is formed from a wire with a diameter approximately equal to the desired inside diameter of the final conduit. Preferably the wire comprises one that will readily neck-down when tension is applied to it (such as a silver plated copper wire) to aid in removal of the final tube. A fitting is slipped over the wire and placed at one end to be incorporated into the construction.

To form a first layer of the expanded PTFE component, a tape of expanded PTFE film is employed comprising a thickness of about 0.01 mm, a width of about 19 mm, radially oriented fibrils with an average fibril length of about 50 microns, a bulk density of about 0.3 g/cc, and a matrix tensile strength of about 90,000 psi (about 620 MPa).

All tensile testing referred to herein was performed at a strain rate of 2 mm/minute/mm under ambient conditions. The fibril lengths of the porous expanded PTFE articles referred to herein are estimated mean values determined by scanning electron photomicrographs.

The expanded PTFE film is helically wrapped in one direction and one pass over the mandrel and fitting with an overlap of the layers of about 50 to 75%. This film layer facilitates removal of the tube from the mandrel.

Next, a second layer is formed using expanded PTFE film coated on one surface with a layer of fluorinated ethylene propylene (FEP) or other impermeable material. The expanded PTFE film has an approximate FEP layer thickness of about 0.0008 mm and a composite thickness of about 0.005 mm, radially oriented fibrils ranging from about 10 to 50 microns in fibril length on the expanded PTFE surface, a combined bulk density ranging from 1.0-2.0 g/cc, and a matrix tensile strength of about 130,000 psi (about 900 MPa).

A preferred FEP-coated expanded PTFE film may be made by a process that comprises the steps of:

a) contacting a porous PTFE substrate, usually in the form of a membrane or film, with another layer which is preferably a film of FEP (about 0.013 mm thick) or alternatively of another thermoplastic polymer;

b) heating the composition obtained in step a) to a temperature above the melting point of the thermoplastic polymer;

c) stretching the heated composition of step b) while maintaining the temperature above the melting point of the thermoplastic polymer; and

d) cooling the product of step c).

One or more wraps (e.g., 2 to 5 wraps or more) of this second film are formed around the tube construction and fitting in a cigarette fashion. “Cigarette wrapping” is defined as circumferentially wrapping a wide sheet of film around the conduit in a manner similar to a rolled cigarette. A continuous FEP coating renders the film, and hence the tube, impermeable when applied in this manner. In the final construct, the presence of the impermeable material serves to prevent cryosurgery fluid from seeping through the conduit during fluid transport.

A third layer identical to the first layer is applied in the same manner to cover the impermeable layer. This layer of expanded PTFE retracts or shrinks when heated.

Next a fourth layer of porous expanded PTFE is formed using an extruded tube of expanded PTFE comprising a wall thickness of about 0.9 mm, a fibril length of about 30 microns, a bulk density of about 0.5 g/cc, and matrix tensile strength of about 20,000 psi (about 140 MPa). The extruded tube is stretched over the film covered mandrel and fitting. This layer serves as an insulation layer.

Finally, a fifth layer identical to the first layer is applied in the same manner to the construction. This layer also retracts when heated.

The mandrel with the multiple expanded PTFE layers attached to it is heated in a convection oven set at about 370° C. for about 6 to 10 minutes, depending on the size and mass of the construction mandrel.

After removing from the oven and cooling, the wire mandrel is longitudinally stretched to reduce its diameter and permit removal of the conduit. The conduit is then cut to a desired length.

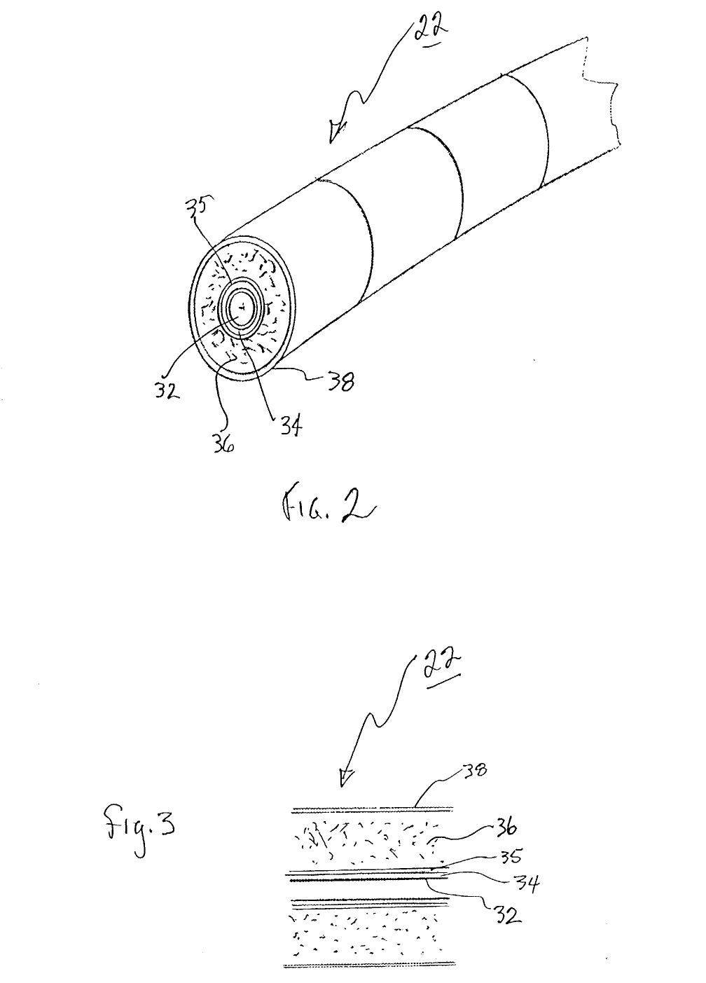

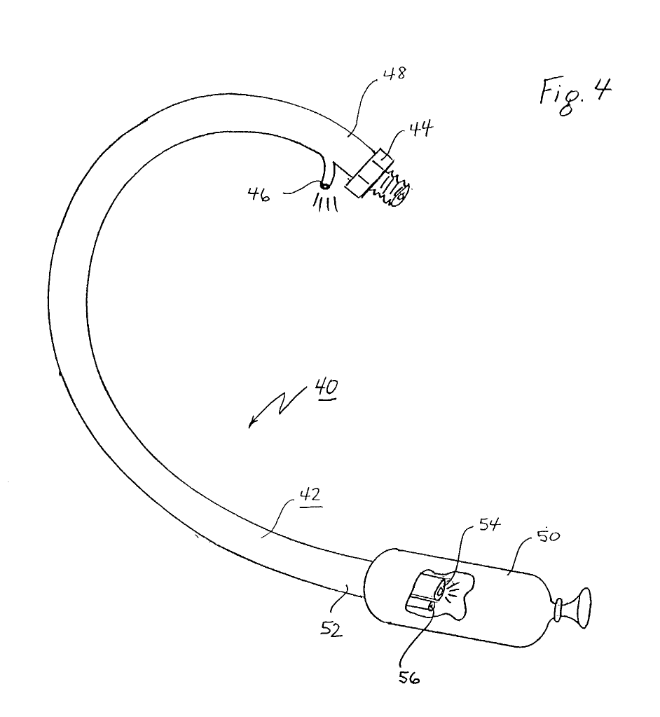

FIGS. 2 and 3 illustrate a conduit 22 made in this manner, comprising the first layer 32, the second impermeable layer 34, the third layer 35, the fourth insulation layer 36, and the fifth layer 38.

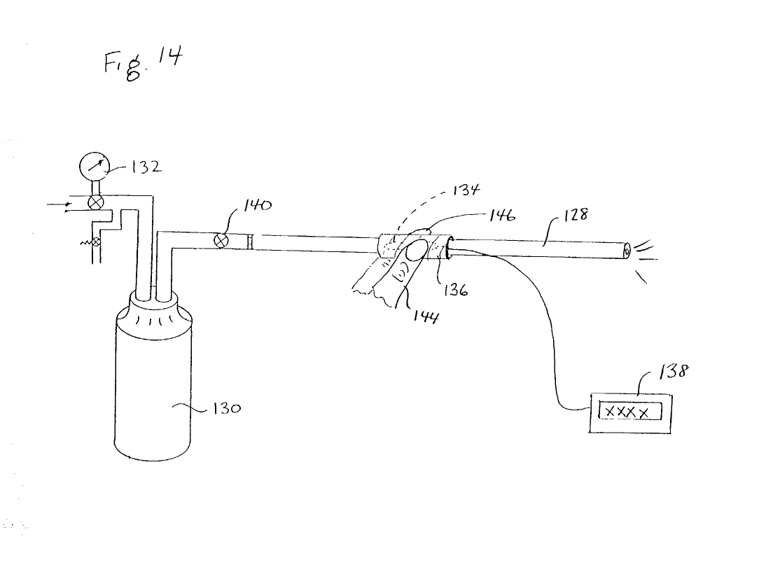

This resulting conduit demonstrates excellent thermal properties. First, the conduit will readily transport liquid nitrogen over a length of tube of at least about 12 inches (about 30 cm). This is demonstrated by delivering liquid nitrogen under 5 psi (about 34 KPa) pressure and liquid nitrogen spraying out the end of the conduit after 5 seconds or less. Second, while delivering liquid nitrogen a conduit with the above construction and a wall thickness of less than about 1 mm and inner diameter of about 1.25 mm has an exterior surface temperature of about 4° C. after one minute of fluid transport. At this temperature the conduit can be handled with bare hands and should not cause tissue damage if the exterior of the tube comes in contact with a patient's tissue for short periods during cryosurgery fluid transport. Third, the conduit is very flexible and a kink can be easily formed to stop the flow of nitrogen. A “flow-stopping kink” can be formed by bending the conduit at an angle sufficient to completely cease fluid flow through the conduit (typically at a bend angle of about 90 to 180 degrees from straight (that is, from the normal, unbent orientation of the conduit)). Once the kink is released, normal liquid nitrogen flow is restored through the conduit without catastrophic damage to the conduit at the site of the kink. Preferably, the conduit of the present invention can withstand a flow-stopping kink with no compromise of the conduit wall integrity and thus no fluid leakage through the conduit wall at the site of the kink. A conduit undergoing such a flow-stopping kink is illustrated in FIG. 13 and described below with reference to the examples.

It should be evident that the number and order of the expanded PTFE layers of the conduit may be modified to address particular design criteria. For example, thicker and/or more layers of material may be included to further insulate the conduit for some applications. Additionally, the FEP coated layer, which, as noted, is provided to make the tube impermeable to cryosurgery fluid penetration, can be oriented in different positions within the conduit wall or may be eliminated from part or all of the construction where seepage or release of cryosurgery fluid through the conduit wall is desired. Additional components, such as wire coils, braids, and/or electrical conductors, may also be included to enhance the utility of the conduit.

It should be further appreciated that the proportions of the conduit may be modified to address specific applications, such as changing the lumen size(s), wall thickness, operating pressures or other conditions, and/or materials to accommodate short-length or long-length delivery of cryosurgery fluids. For example, conduits of the present invention may be made with lengths of about 6 inches (about 15 cm), about 18 inches (about 45 cm), about 24 inches (about 60 cm), about 36 inches (about 90 cm), or more. Additionally, the conduit may be formed into different cross-sectional shapes, such as circular, oblong, rectangular, etc. Further, the conduit may be formed with varying dimensions along its length, such as being formed with tapers, steps, ribs, braids, etc.

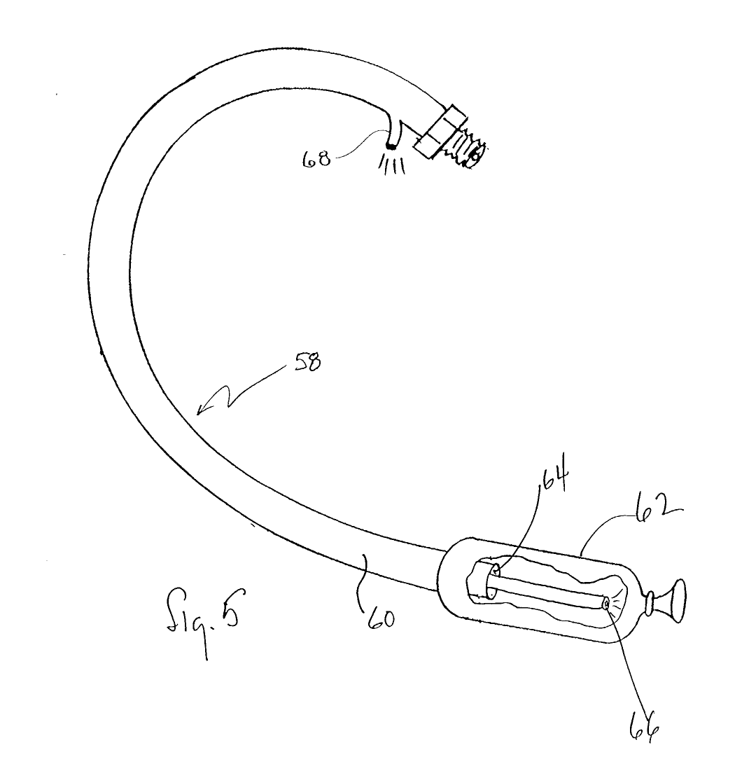

FIG. 4 illustrates another embodiment of delivery apparatus 40 of the present invention. In this embodiment, the apparatus 40 comprises a dual lumen conduit 42, having a fluid source fitting 44 and an exhaust port 46 on a first end 48 of the conduit, and a treatment instrumentality 50 in the form of a thermally conductive balloon or probe on a second end 52 of the conduit. As is shown in the cut-away on the treatment instrumentality 50, the second end 52 of the conduit 42 includes a delivery port 54 and a fluid return port 56.

A dual lumen construction allows the fitting 44 to be attached to a cryosurgery fluid source with cryosurgery fluid being delivered to the treatment instrumentality 50 through the delivery port 54, while fresh flow of fluid through the apparatus 40 is maintained by allowing cryosurgery fluid to exit the instrumentality through the fluid return port 56 and be released safely away from the patient through the exhaust port 46. By delivering cryosurgery fluid in this manner, the treatment instrumentality can be maintained at a very low and consistent cryosurgery temperature throughout treatment, and cryosurgery fluid is safely removed from the patient so as to avoid release and possible complications at the treatment site.

For use with the present invention, the treatment instrumentality may take a variety of forms. In its simplest form, the treatment instrumentality may comprise a simple open-end or nozzle on the end of the conduit that provides controlled release of cryosurgery fluid at the treatment site. Further, as is shown in FIG. 4, the treatment instrumentality may comprise an inflatable structure, such as a balloon constructed from a porous or impermeable material, that is thermally conductive. In the embodiment shown in FIG. 4, the instrumentality 50 comprises a balloon formed from an impermeable polymer, such as FEP, that presents a very cold surface for cryosurgical treatment at the treatment site, without permitting release of cryosurgery fluid into the patient. Additionally, as is described below, other treatment instrumentalities that may be employed with the present invention may include: impermeable probes, such as ones constructed from metal, plastic, glass, composite materials, etc.; permeable probes allowing for controlled release of cryosurgery fluid at the treatment site; open-end or closed-end needles that permit penetration into target tissue, with or without cryosurgery fluid release. Due to the layered construction it is also possible to incorporate conductors in the conduit wall for temperature monitoring, thawing heaters and the like.

The apparatus 58 shown in FIG. 5 illustrates another embodiment of a conduit 60 and treatment instrumentality 62 that can be used with the present invention. In this embodiment, an annular space 64 is created between coaxially positioned conduits 60 and 66. Various treatment instrumentalities 62 may be included, such as that previously described in FIG. 4, above. The annular space 64 serves both to assist in insulating the exterior of the tube from the inner conduit 66 and also to serve as a return conduit to allow fluid to exit through exhaust port 68. Additional advantages of this construction include enhanced bending characteristics and an overall lower diameter profile.

The inventive apparatus 70 illustrated in FIG. 6 comprises two separate conduits 72, 74 attached, respectively, to inlet and outlet fittings 76, 78 on a treatment instrumentality 80 in the form of a probe. Conduit 72 connects between a cryosurgery fluid source and the inlet fitting 76 to provide for delivery of cryosurgery fluid to the probe 80. Conduit 74 connects between the outlet fitting 78 and an exhaust port (not shown) to provide for removal of cryosurgery fluid from the treatment site. The advantage of this construction is that it allows for use of easier-to-construct and lower profile single lumen tubes that may be cheaper to use and may provide greater flexibility in use. Additionally, by providing a completely separate return conduit, the medical personnel may be provided with more options on where cryosurgery fluid can be discharged.

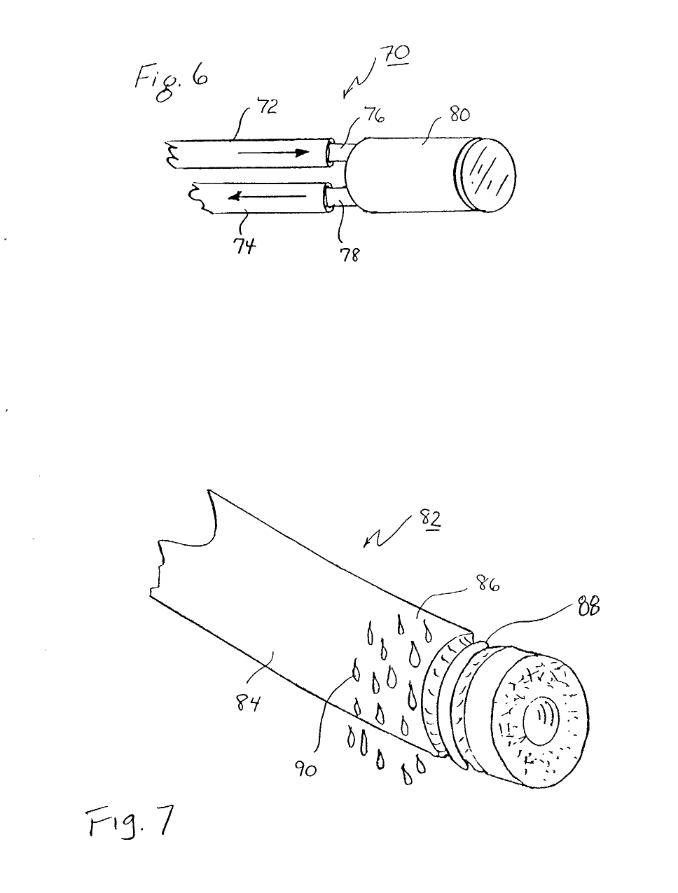

Shown in FIG. 7 is still another embodiment of an apparatus 82 of the present invention. In this embodiment, a conduit 84 is employed that is permeable to cryosurgery fluid at its second end 86. An end tie or clamp 88 is employed to seal the end of the conduit. In operation, the cryosurgery fluid will seep out of the end 86 of the conduit in a controlled manner to provide precise cryosurgery fluid delivery, as is demonstrated with the droplets of cryosurgery liquid 90 shown in FIG. 7.

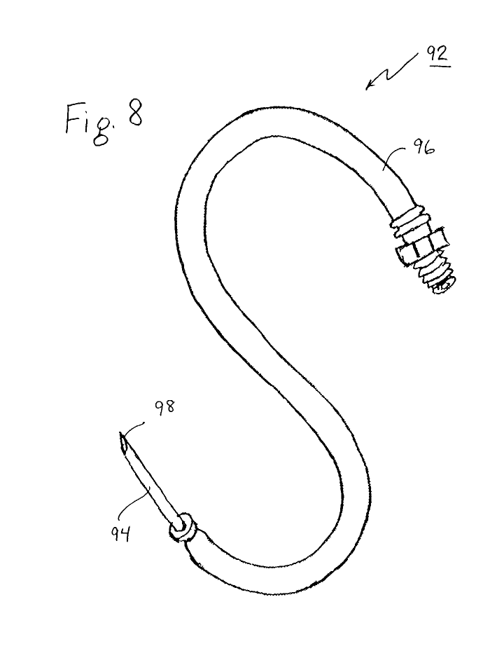

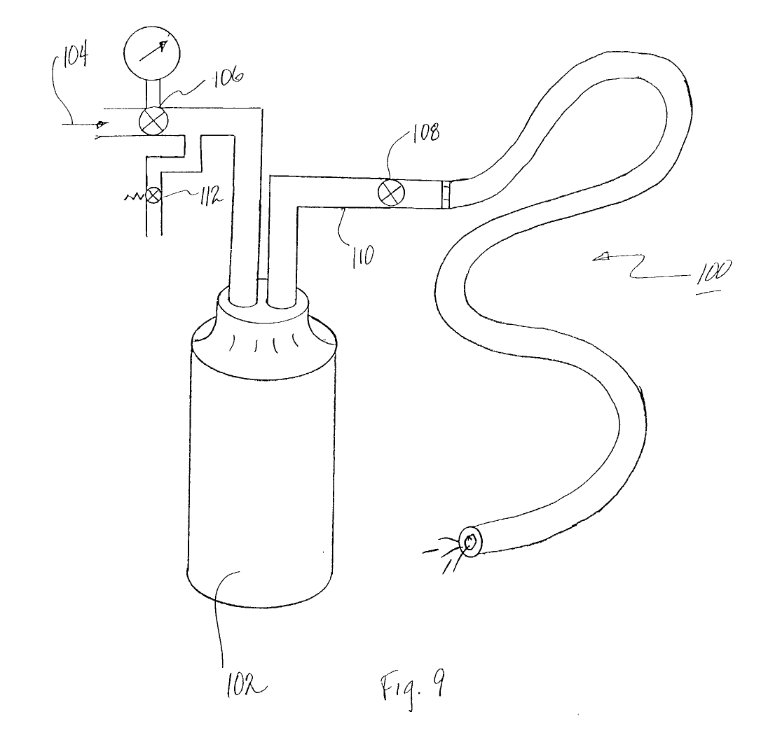

FIG. 8 shows an embodiment of the apparatus 92 of the present invention that employs a needle 94 at the end of the conduit 96. The needle 94 includes a sharp end that allows the needle to be inserted into targeted tissue for exact cryosurgery treatment. The needle may include one or more openings 98 therein to permit cryosurgery fluid to directly contact the targeted tissue. Alternatively, the needle may be sealed at its end, without or without a cryosurgery fluid return lumen or conduit (as has been described with respect to FIGS. 4, 5, and 6), to allow targeted treatment without cryosurgery fluid release. Open or closed, suitable needles may be formed from metal, plastic, glass, or other material, as appropriate for a given procedure.

FIG. 9 shows schematically delivery apparatus 100 of the present invention attached to a dewar 102 containing cryosurgery fluid. Suitable dewars for use with the present invention are available from a number of sources, including Brymill Cryogenics Systems, Ellington, Conn. The dewar 102 should be pressurized using a pressure source 104 and regulator valve 106. Suitable pressure may be generated using conventional pressure pump apparatus, such as an air compressor available from Gast Manufacturing, Benton Harbor, Mich. A valve 108 is provided on the delivery line 110 connected between the dewar 102 and the delivery apparatus 100 to allow the medical staff to control release of cryosurgery fluid. Additionally, a safety valve 112 may be provided between the pressure source 104 and the dewar 102 to avoid over-pressurization of the dewar.

One of the advantages of the apparatus of the present invention is that its exceptional thermal efficiency allows it to deliver cryosurgery fluid at much lower pressures than those normally employed with existing fluid delivery apparatus used in surgical procedures. By contrast to the present invention, some systems use high-pressure fluids such as gaseous nitrogen or argon. Typically fluid delivery through these devices require 3000 psi (about 20 MPa) of pressure supplied through a supply line into a heat exchanger and cooling fluid outlet and Joule-Thompson nozzle. Expansion of the high-pressure gas cools the instrumentality.

The present invention can deliver cryosurgery liquid at pressures below about 50 psi (about 345 KPa) and more preferably below about 20 psi (about 140 KPa) and most preferably below about 15 psi (about 100 KPa). The ability to reliably deliver cryosurgery liquid or gas at much lower pressures provides numerous advantages, including allowing more controlled fluid delivery, providing a much safer environment in the event of equipment failure, and allowing fluid to be delivered with smaller and less expensive apparatus (e.g., smaller and simpler pumps and dewars).

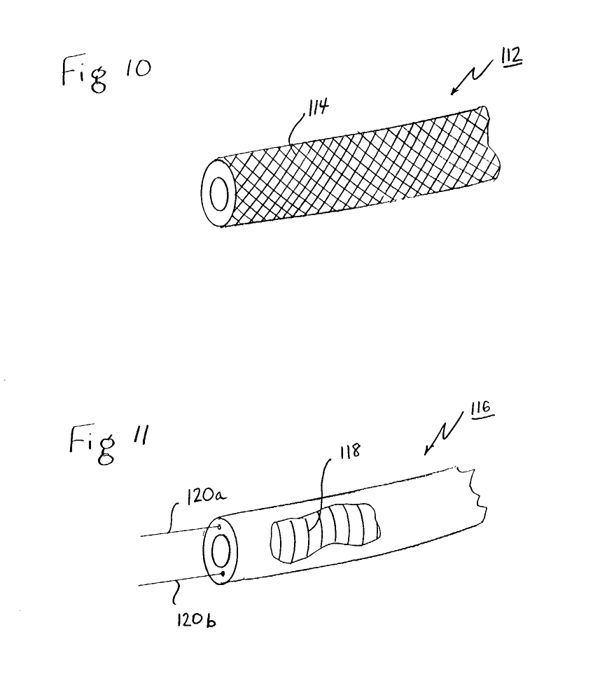

As has been noted, the present invention can be readily adapted to provide additional functions for cryosurgery procedures, such as providing wire coils, braids, and/or electrical conductors. FIG. 10 illustrates a conduit 112 of the present invention that further includes a braided cover 114 around its circumference. The cover 114 provides a simple means of improving the insulative qualities of the conduit and/or its ability to be handled. Such a cover may be constructed from a variety of materials, including ceramic, glass, metal or polymer, and may take a variety of forms, including braids, ribs, rings, helixes, coils, felts, etc.

FIG. 11 illustrates a conduit 116 of the present invention that further includes embedded electrical conductors 118, with leads 120a, 120b provided for electrical connections. Such a conductors can be provided to allow for electrical feedback of information from the conduit and/or the instrumentality, such as with use of a thermocouple or other sensing device, or the conductor 118 may also be used to provide for selective heating of the conduit and/or the instrumentality when desired, such as with the heating coil shown.

The apparatus of the present invention may be used with any form of cryosurgery fluid, including without limitation liquid or gaseous: nitrogen, oxygen, air, argon, helium, etc. Additionally, the apparatus of the present invention can be employed in virtually any form of medical procedure, including without limitation: topical skin treatments (such as, dermatology treatments for skin cancer); open or endoscopic surgical procedures, such as those for tachyarrhythmia; treatment of abnormal cell growth of various organs, such as kidneys, breasts, lungs, prostates, and livers; endoluminal procedures, such as treating stenosis and other vascular pathologies; neurologic applications, such as performing nerve ablation; hypothermic treatments; etc.

Without intending to limit the present invention to the specifics described hereinafter, the following examples illustrate how the present invention may be made.

### EXAMPLE 1

A first comparative example using a non-porous fluoropolymer tube was tested. Fluorinated ethylene propylene (FEP) tubing (density of about 2.1 g/cc); 0.053″ (1.4 mm) internal diameter; 0.016″ (0.41 mm) wall thickness) was obtained from Zeus Industrial Products Inc., Raritan, N.J. A 12″ (305 mm) length was fitted with a small 10-32 threaded brass barb fitting available from Clippard Instrument Laboratory, Inc., Cincinnati, Ohio. The fitting was inserted into one end with the aid of a heat gun and secured with a small wire tie.

This example and all subsequent examples were tested for liquid cryogen delivery characteristics by connecting to a pressurized dewar (vacuum insulated bottle) of liquid nitrogen, such as in the apparatus shown and described with respect to FIG. 9. The dewar was obtained from Brymill Cryogenic Systems, Ellington, Conn., and was connected to a compressed air source and a precision pressure regulator.

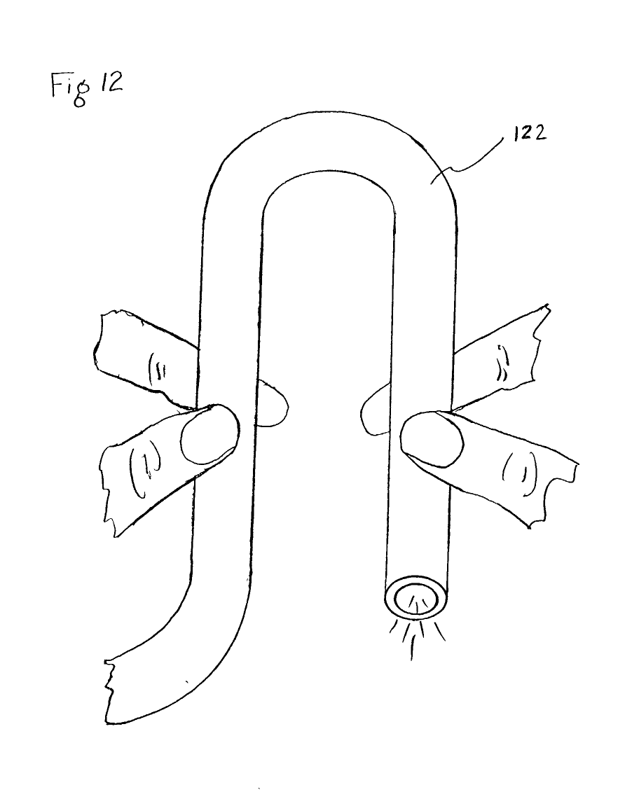

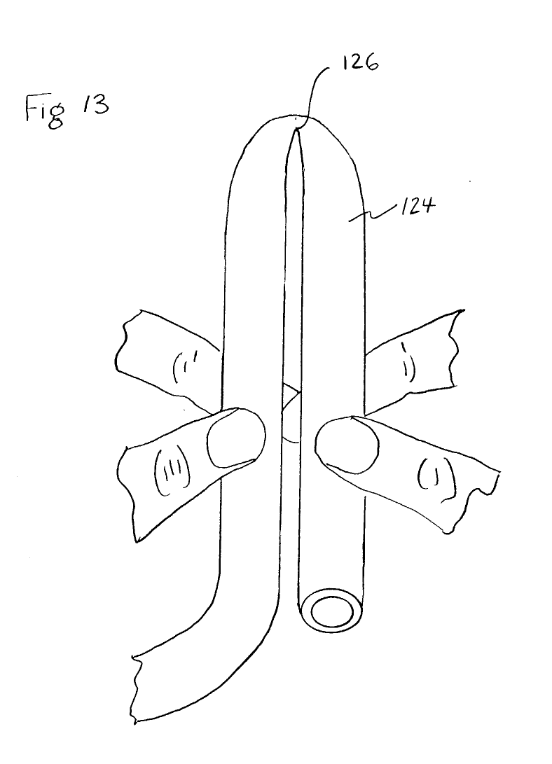

The tube was held in a straight condition and, at a 5 psi (about 35 KPa) pressure setting, the tube was charged with liquid nitrogen by opening a valve connected to the liquid dip tube within the dewar. Liquid nitrogen sprayed out the end within 1-2 seconds, as confirmed by wetting of an expanded PTFE sheet held in front of the liquid stream. Next the tube was bent to attempt to kink and interrupt the flow of liquid nitrogen. The tube was grasped in two places with about 4 inches (102 mm) of tube exposed and bent to form an arc with a radius of curvature of approximately 8 mm. A generic tube 122 in this starting condition is illustrated in FIG. 12. The FEP tube failed catastrophically by snapping into two separate pieces at a bend angle of approximately 180 degrees off straight, with approximately an 8 mm radius of curvature, before a kink sufficient to cease fluid flow could be formed. The outer surface was also very cold and required gloves to prevent a cold-burn while handling.

### EXAMPLE 2

A second comparative example comprising non-expanded, non-porous polytetrafluoroethylene (PTFE) tubing (density of about 2.2 g/cc; 0.053″ (1.4 mm) internal diameter; 0.016″ (0.41 mm) wall thickness) was obtained from Zeus Industrial Products, and a repeat of the test of Example 1 was performed. Results were identical with liquid nitrogen spraying out the end within 1-2 seconds. This sample was bent like the tube in Example 1 and catastrophic breakage occurred at a bend angle of approximately 180 degrees off of straight, with approximately 25 mm radius of curvature. The surface was also cold and required gloves to prevent a cold-burn while handling.

### EXAMPLE 3

A third comparative example was tested comprising a piece of stainless steel needle tubing (0.05″ (1.3 mm) internal diameter and 0.006″ (0.15 mm) wall thickness) available from The Microgroup Inc., Medway, Mass. A brass barb fitting was soldered to the end of a 12″ (305 mm) length to connect to the dewar. The dewar was pressurized to 5 psi (about 35 KPa) and when the valve was opened liquid nitrogen sprayed out the end of the tube within 1-2 seconds. Minimal bending was attempted because of the rigid nature of the material. The surface was extremely cold and required gloves for handling. Breakage of this material would be anticipated even at a large radius of curvature.

### EXAMPLE 4

A first polymer conduit of the present invention as constructed in the following manner:

1. Silver plated copper wire 0.05″ (1.3 mm) in diameter was obtained from Hudson International, N.Y., and used as a construction mandrel. A brass barb fitting was slipped over the wire and placed at one end to be incorporated into the construction.

2. A 0.75″ (19 mm) wide tape of expanded PTFE film, comprising a thickness of about 0.01 mm, radially oriented fibrils with a length of about 50 microns, a bulk density of about 0.3 g/cc, and a matrix tensile strength of about 90,000 psi (620 MPa) was helically wrapped by hand in one direction over the mandrel and brass fitting with about 60% of overlap of the layers. Only one pass of film was applied.

3. An extruded tube of expanded PTFE, comprising a wall thickness of about 0.9 mm, an average fibril length of about 30 microns, a bulk density of about 0.5 g/cc, an internal diameter of about 1.25 mm, and a matrix tensile strength of about 20,000 psi (138 MPa) was stretched over the film covered mandrel and fitting.

4. A film of expanded PTFE coated with a continuous coating of FEP comprising a combined thickness of about 0.0046 mm (0.0038 mm of which is the expanded PTFE film), a combined bulk density ranging from about 1.0-2.0 g/cc, and a matrix tensile strength of about 130,000 psi (897 MPa) was wrapped around the tube construction by hand in a cigarette fashion. The fibril length of the expanded PTFE can be measured by SEM to examine the expanded PTFE side of the composite membrane. The expanded PTFE material had radially oriented fibrils, with fibril lengths of between 10 and 50 microns. Approximately 5 wraps of this material were applied over the mandrel and fitting with the FEP side placed inward facing the mandrel.

5. A wrapping of expanded PTFE film identical to step 2 was applied as a final layer in the same manner.

6. The construction was heated in a convection oven set at 370° C. for 6 minutes.

7. After removal from the oven and cooling the silver plated copper mandrel was longitudinally stretched to reduce its diameter and permit removal of the tube sample. The tube was cut to a 12″ (305 mm) length to form an inventive conduit.

The conduit was held straight and tested with liquid nitrogen in the same manner as the previous Examples. At 5 psi (34.5 KPa) liquid nitrogen sprayed out the end in 3-4 seconds and although very cold was able to be held with bare hands while performing the kink test. The conduit was grasped as described in Example 1 and bent 180 degrees into an arc with the ends parallel to each other, similar to that illustrated in FIG. 12. By gradually moving the parallel ends toward each other the bend radius reduced in size until the conduit yielded into a flow-stopping kink. The conduit 124 with a flow-stopping kink 126 is illustrated in FIG. 13. The radius at that point was approximately 20 mm. When the tube was straightened flow was restored, however, a slight plume of condensation was observed at the kink region indicating a small breach of the conduit wall.

### EXAMPLE 5

A second polymer conduit of the present invention was constructed in the following manner:

1. Silver plated wire as described in Example 4 was used as the construction mandrel. A brass barb fitting was also incorporated in the construction as before.

2. A 0.75″ (19 mm) wide expanded PTFE film as described in step 2 of Example 4 was applied as described in that example.

3. 5 layers of FEP coated expanded PTFE film described in step 4 of Example 4 were applied in the same manner as in Example 4.

4. A second layer of film like that described in step 2 of this example was applied to the construction in the same manner as step 2.

5. An extruded PTFE tube as described in step 3 of Example 4 was stretched over the mandrel and fitting.

6. A final wrapping of the film described in step 2 of this example was applied in the same manner.

7. The construction was heated for in an oven set to 370° C. for 6 minutes, cooled, removed, and cut to a 12″ (305 mm) length as before to form an inventive conduit. This construction is illustrated in FIGS. 2 and 3.

Testing was identical to Example 4 with the pressure set at 5 psi (34.5 KPa). Liquid nitrogen sprayed out the end of the conduit in approximately 1-2 seconds. The conduit was comfortable to handle with bare hands, had good flexibility, and was kink tested in the same manner as Example 4. The conduit also yielded into a kink at approximately a 20 mm radius stopping the flow of nitrogen. When the conduit was straightened to restore the flow of fluid there was no evidence of leaking from in the conduit wall at the site of the kink.

### EXAMPLE 6

A third inventive polymer conduit of the present invention was constructed in the following manner:

1. Silver plated copper wire 0.033″ (0.84 mm) in diameter was used as a construction mandrel.

2. A helical wrapping of expanded PTFE film identical to the film in step 2 of example 4 was applied in the same manner.

3. A cigarette wrapping of FEP coated expanded PTFE film was applied as in step 4 of Example 4

4. A third layer identical to step 2 of this example was applied to the construction.

5. A helical wrapping of 0.75″ (19 mm) wide expanded PTFE film comprising a thickness of about 0.0015″ (0.038 mm), substantially longitudinally oriented fibrils with a length ranging from about 100-300 microns, a bulk density ranging from about 0.1-0.2 g/cc, and a matrix tensile strength of about 25,000 psi (172 MPa) was applied with about 60% overlap of the layers. A total of 5 passes were applied in alternating directions.

6. A final layer identical to step 2 was applied to the construction.

7. The construction was heated in an oven set at 370° C. for 6 minutes, cooled, removed and cut to a 12″ (305 mm) length as before to form an inventive conduit.

Testing was performed as before but the dewar was pressurized to 30 psi (207 KPa). Liquid nitrogen sprayed from the conduit after about 3 seconds. The conduit was kink tested as in the previous two examples. A flow-stopping kink occurred at about 16 mm radius. When the conduit was straightened to restore the flow of fluid, there was no evidence of leaking from the conduit wall at the site of the kink.

### EXAMPLE 7

An inventive 12″ (305 mm) polymer conduit was constructed in accordance with Example 5 and fitted with a needle to form a simple cryogenic catheter with a delivery instrumentality at the distal end. An abrasive saw was used to cut the hub off of a standard 16 gauge syringe needle and the shaft end was then inserted into the end of the example conduit for about 0.4″ (10.2 mm). The needle was secured with a string tie of expanded PTFE sewing thread. This construction is illustrated in FIG. 8.

The dewar was pressurized to 5 psi (34.5 KPa) and the conduit was flow tested in the manner previously described in the other examples. Liquid nitrogen sprayed out of the needle in approximately 1-2 seconds.

### EXAMPLE 8

A conduit of the present invention was constructed to demonstrate another means of delivering cryogenic liquid via polymer catheter tubing. A 12″ (305 mm) conduit of Example 5 was constructed but the FEP coated expanded PTFE film was not applied for a distance of about 1″ (25 mm) at the distal end. Next, a string tie of expanded PTFE sewing thread was placed about 0.1 ″ (2.5 mm) from the distal end to close the conduit completely. This construction is illustrated in FIG. 7.

The conduit was flow tested as in the other examples but the dewar was pressurized to 10 psi (69 KPa). In approximately 3 seconds drops of liquid nitrogen flowed from the distal 1″ (25 mm) length of the conduit demonstrating a more controlled delivery of liquid cryogen. No spraying of liquid nitrogen was observed from the conduit surface. The kink test was performed as before. A flow-stopping kink occurred at about a 20 mm radius. When the conduit was straightened to restore fluid flow, there was no evidence of leaking from the conduit wall at the site of the kink.

### EXAMPLE 9

This example was constructed to demonstrate a closed-loop liquid cryogenic catheter with a metal probe. Two 12″ (305 mm) conduits of Example 5 were attached to a stainless steel probe in the manner shown in FIG. 6. One conduit delivered the liquid nitrogen to the probe and the other was used to vent the probe to provide liquid cryogen flow. The probe was constructed from two 0.47″ (12 mm) diameter stainless tubes and one larger 0.093″ (2.4 mm) diameter stainless tube (available from The Micro Group, Inc., Medway, Mass.) by soldering the two smaller tubes into one end of the larger tube, forming a “Y”. The large tube open end was then sealed off with a stainless steel set screw. Finally the two conduits of Example 5 were attached onto the small stainless steel tubes of the probe “Y” by an expanded PTFE sewing thread tie. This construction is illustrated in FIG. 6.

Testing was the same as in other Examples with the dewar pressurized to 20 psi (138 KPa). One conduit was connected to the dewar dip tube opening and the other conduit was open to atmosphere. At approximately 3 seconds the liquid nitrogen traveled down one conduit, cooled the metal probe, and sprayed liquid out of the open end of the second conduit.

### EXAMPLE 10

A second closed-loop catheter example was constructed to demonstrate a method of delivering liquid cryogen to a polymer balloon. As in Example 9, one conduit supplied liquid cryogen to the balloon and one conduit was a fluid vent allowing liquid cryogen to exit the balloon.

The polymer catheter was constructed in the following manner:

1. A tube of Example 5 with a brass fitting and a tube of Example 6 without a brass fitting were used for the construction (note the mandrels were not removed until the last step because of subsequent added layers described in step 4 and 5, and the final heating step 6.

2. A 6 mm diameter stainless tube approximately 2″ (51 mm) long was used as a mandrel to form the balloon. The two tubes of step one were held side-by-side and inserted through the stainless tube with about 2 cm extending out of the end.

3. One pass of expanded PTFE film of step 2 of Example 4 was wrapped onto the stainless tube portion of the construction with about 60% overlap.

4. An expanded PTFE/FEP film of Example 4 step 4 was wrapped around both of the tubes held side-by-side and the balloon mandrel in a cigarette fashion. About 5 layers were applied leaving the ends of the polymer tubes open to create one dual lumen conduit.

5. A helical wrapping of expanded PTFE film was applied as in step 2 Example 4 to the entire construction.

6. The construction was heated in an oven set at 370° C. for 10 minutes, cooled and removed from the wire mandrels. The stainless balloon mandrel was also removed leaving a flexible fluid tight perfluoropolymer balloon about 0.0017″ (0.043 mm) thick.

7. To finish the balloon end of the catheter, the balloon portion was pulled back exposing the two ends of the dual lumen catheter conduit. The two ends were trimmed to approximately ⅓ of the balloon length.

8. The balloon material was then pulled past the short catheter ends and tied off at the end with expanded PTFE sewing thread. The final length of the device was about 12″ (305 mm) in length. An illustration of this construction is shown in FIG. 4.

Testing was performed as before with the dewar pressure set at 40 psi (276 KPa). The larger catheter conduit with the brass fitting was the supply tube and connected to the dewar. The smaller conduit was the vent and left open to atmosphere. Liquid nitrogen entered the balloon and sprayed out the vent in about 50 seconds.

It should be appreciated that a variety of perfluoropolymer balloons in accordance with this example may be created as instrumentalities for use with the present invention. For instance, the thickness of the perfluoropolymer balloon may range from about 0.01 to about 0.1 mm, and more preferably from about 0.02 to about 0.08 mm. Additionally, the non-permeable fluoropolymer layer may be formed from a variety of materials in addition to or in place of FEP, such as perfluoroalkoxy polymer (PFA), tetrafluoroethylene (TFE), ethylene-tetrafluoroethylene (ETFE), etc.

### EXAMPLE 11

A test of the thermal properties of a tube of the present invention as compared with other tubes was performed in the following manner using the test apparatus schematically represented in FIG. 14:

1. Tubes 128 in accordance with Examples 1, 2, 3, and 5 were tested to determine their relative thermal qualities. All testing was performed in air at ambient room temperature (23° C.).

2. A dewar 130 (Brymill Cryogenic Systems, Ellington, Conn.) charged with liquid nitrogen (at below −150° C.) was connected to a compressed air source and a precision pressure regulator 132 and pressurized to 15 psi (about 105 KPa). Each tube 128 was in turn attached to the dewar 130.

3. A thermocouple 134 (K-Type Thermocouple from Omega Engineering, Inc., Stamford, Conn.) was attached at approximately the mid-point on each tube 128 during the test. The thermocouple 134 was placed along the tube 128 and then wrapped in place using about five layers of expanded PTFE tape 136, approximately 0.01 mm thick, so as to hold the thermocouple 134 in contact with the tube 128 during the test. The thermocouple 134 was attached to a multifunction calibrator 138 (Model TRC-82, from Wahl Instruments Inc., Asheville, N.C.) in order to record temperature readings.

4. Each tube 128 was then charged with liquid nitrogen by opening a valve 140 connected to the liquid dip tube 142 within the dewar 130. In all cases, liquid nitrogen sprayed out the end within 1 second, as confirmed by wetting of an expanded PTFE sheet held in front of the liquid stream.

5. After liquid nitrogen sprayed out of the end of each tube 128 for 10 seconds, a temperature reading was taken and recorded.

6. The test was then repeated for each tube 128 with a tester's thumb 144 and forefinger 146 holding the outside of the wrapped thermocouple 134, as is shown in FIG. 14, in order to simulate the effect of heat absorption by a human body. Temperature readings were taken after 5 seconds of liquid nitrogen spray.

The test results are summarized in the following table:

| | Time to | Temp. @ 10 Sec. | Temp. @ 5 Sec. |
|---|---|---|---|
| Sample Tested | Liquid Spray | Open Air | Held by Fingers |
| FEP Tube | 1 second | −117° C. | −2° C. |
| (Example 1) | | | |
| Non-Porous PTFE | 1 second | −127° C. | −13° C. |
| Tube (Example 2) | | | |
| Stainless Steel | 1 second | −160° C. | −36° C. |
| Tube (Example 3) | | | |
| Inventive Tube | 1 second | −69° C. | +19° C. |
| (Example 5) | | | |

The test demonstrates the vastly improved insulative properties of the tube of the present invention as compared with prior art tubes used to carry cryogenic fluids.

While particular embodiments of the present invention have been illustrated and described herein, the present invention should not be limited to such illustrations and descriptions. It should be apparent that changes and modifications may be incorporated and embodied as part of the present invention within the scope of the following claims.

The invention claimed is:

1. A medical apparatus comprising
a flexible conduit of expanded polytetrafluoroethylene;
a fitting on the conduit adapted to attach the conduit to a source of liquid cryosurgery fluid; and
an instrumentality on the conduit adapted to employ the cryosurgery fluid at a treatment site;
wherein the conduit is adapted to convey cryosurgery fluid from the source of liquid cryosurgery fluid to the instrumentality.

2. The medical apparatus of claim 1 wherein the conduit includes a wall and the conduit is capable of being bent at least 45 degrees from normal plane while filled with a fluid having a temperature below negative 80° C. without leaking through the wall.

3. The medical apparatus of claim 1 wherein the apparatus further includes a second conduit to remove cryosurgery fluid from the treatment site.

4. The medical apparatus of claim 3 wherein the second conduit comprises expanded polytetrafluoroethylene.

5. The medical apparatus of claim 1 wherein the flexible conduit includes at least two lumens through at least a portion thereof.

6. The medical apparatus of claim 1 wherein the instrumentality comprises a sealed probe.

7. The medical apparatus of claim 1 wherein the instrumentality comprises a probe having at least one opening therein to allow for the release of cryosurgery fluid.

8. The medical apparatus of claim 7 wherein the probe includes a sharpened end allow the probe to serve as a penetrating needle.

9. The medical apparatus of claim 1 wherein the instrumentality comprises a permeable material allowing the release of cryosurgery fluid.

10. The medical apparatus of claim 1 wherein while conveying cryosurgery fluids at a temperature of less than negative 80° C. the flexible conduit can be bent to form a flow-stopping kink at a kink site and then straightened to return normal fluid flow through the conduit without leakage of cryosurgery fluid from the conduit at the kink site.

11. The medical apparatus of claim 1 wherein the cryosurgery fluid comprises liquid air.

12. The medical apparatus of claim 1 wherein the cryosurgery fluid comprises liquid nitrogen.

13. The medical apparatus of claim 1 wherein the conduit has a wall and cryosurgery fluid does not appreciably leak through the conduit wall during treatment.

14. The medical apparatus of claim 1 wherein the conduit comprises two tubes mounted coaxially with each other.

15. A method of performing surgery comprising
providing a conduit formed from expanded PTFE, the conduit having a wall and being capable of being bent at least 90 degrees from a straight orientation without leaking through the wall while filled with a cryosurgery fluid with a
temperature of less than negative 80° C., the conduit including a distal end capable of delivering cryosurgery fluid to a treatment site;
providing a cryosurgery fluid source;
connecting the conduit to the cryosurgery fluid source;
delivering cryogenic fluid from the cryosurgery fluid source through the conduit to the treatment site.

16. The method of claim 15 that further comprises
providing a conduit that includes at least two lumens therethrough;
providing a sealed treatment instrumentality;
connecting a first lumen in the conduit between the cryosurgery fluid source and the treatment instrumentality, and connecting a second lumen in the conduit between the treatment instrumentality and an exhaust port;
delivering cryosurgery fluid from the fluid source through the first lumen so as to cool the treatment instrumentality, and exhausting cryosurgery fluid from the treatment instrumentality through the second lumen and the exhaust port.

17. A flexible polymer conduit comprising
a polymer conduit having an internal diameter of less than about 2.5 mm (0.1 inch);
the conduit being capable of conveying liquid cryosurgery fluid without leaking; and
the conduit being capable of being kinked to stop the flow of liquid cryosurgery fluid without breaking.

18. A catheter for delivery of a cryosurgery fluid to a treatment site comprising
a flexible conduit having a first end and a second end;
wherein the first end is adapted for attachment to a cryosurgery fluid source; and
wherein the second end is adapted to deliver cryosurgery fluid to the treatment site; and
wherein while conveying cryosurgery fluids at a temperature of less than negative 80° C. the conduit can be bent to form a flow-stopping kink at a kink site and then straightened to return fluid flow through the conduit without leakage of cryosurgery fluid from the conduit at the kink site.

19. The catheter of claim 18 wherein the conduit is capable of being bent to form a flow-stopping kink at a kink site and then straightened to return fluid flow through the conduit without leakage of cryosurgery fluid from the conduit at the kink site while conveying cryosurgery fluid at a temperature below negative 150° C.

20. The catheter of claim 18 wherein the cryosurgery fluid comprises liquid nitrogen.

21. The catheter of claim 18 wherein the catheter further includes a second conduit to remove cryosurgery fluid from the treatment site.

22. The catheter of claim 21 wherein a sealed instrumentality is provided on the second end of the conduit.

23. The catheter of claim 22 wherein the sealed instrumentality comprises a probe.

24. The catheter of claim 23 wherein the probe includes a sharpened end allow the probe to serve as a penetrating needle.

25. The catheter of claim 18 wherein the conduit comprises expanded polytetrafluoroethylene.

26. The catheter of claim 23 wherein the flexible conduit includes at least two lumens through at least a portion thereof.

27. The catheter of claim 18 wherein the cryosurgery fluid comprises liquid air.

28. The catheter of claim 18 wherein the conduit includes a wall and cryosurgery fluid does not appreciably leak through the conduit wall during treatment.

29. The catheter of claim 18 wherein the conduit comprises two tubes mounted coaxially with each other.

30. The catheter of claim 18 wherein the conduit comprises an expanded polytetrafluoroethylene.

31. The catheter of claim 30 wherein the conduit includes at least one additional polymer.

32. The catheter of claim 31 wherein the additional polymer comprises FEP.

33. The medical apparatus of claim 1 wherein the conduit includes at least one additional polymer.

34. The medical apparatus of claim 30 wherein the additional polymer comprises FEP.

35. The medical apparatus of claim 1 wherein the flexible conduit includes variable permeability along its length.
